(19) United States
(12) Patent Application Publication
(10) Pub. No.: US 2025/0043082 A1
(43) Pub. Date: Feb. 6, 2025

(54) CROSSLINKING AGENTS AND MEDICAL HYDROGELS FORMED THEREFROM

(71) Applicant: Boston Scientific Scimed, Inc., Maple Grove, MN (US)

(72) Inventors: Nicolas Ball-Jones, Minneapolis, MN (US); Yen-Hao Hsu, Shrewsbury, MA (US); Sasha Viola Rios, Minneapolis, MN (US); Joseph Thomas Delaney, JR., Minneapolis, MN (US); Rick Dorn, Plymouth, MN (US); Cristian Parisi, Boston, MA (US)

(73) Assignee: Boston Scientific Scimed, Inc., Maple Grove, MN (US)

(21) Appl. No.: 18/781,773

(22) Filed: Jul. 23, 2024

Related U.S. Application Data
(60) Provisional application No. 63/515,513, filed on Jul. 25, 2023.

(51) Int. Cl. C08J 3/075; A61K 31/74; A61K 38/02; C07K 1/14; C08G 69/04

(52) U.S. Cl. CPC C08J 3/075; A61K 31/74; A61K 38/02; C07K 1/14; C08G 69/04

(57) ABSTRACT

In some aspects, the present disclosure pertains to methods that comprise (a) performing a ring-opening polymerization of one or more types of amino acid N-carboxyanhydride (NCA) monomers that comprise at least one type of protected amino acid NCA monomer having a protected pendant amine group in the presence of an initiator compound to produce intermediate peptide compounds that comprise an amino acid chain having protected pendant amine groups covalently attached to a residue of the initiator and (b) deprotecting the intermediate peptide compounds to form final peptide compounds that comprise an amino acid chain having pendant amine groups covalently attached to the residue of the initiator.

### CROSS-REFERENCE TO RELATED APPLICATIONS

This application claims the benefit of U.S. Provisional Patent Application Ser. No. 63/515,513 filed on Jul. 25, 2023, the disclosure of which is incorporated herein by reference.

### FIELD

The present disclosure relates to methods of forming peptides from amino acid N-carboxyanhydride monomers using a variety of initiators, including methods of forming radiopaque peptides using radiopaque initiators. The present disclosure also relates to the use of such peptides as crosslinking agents for forming hydrogels, and to hydrogels formed from such peptides. The crosslinking agents and hydrogels are useful, for example, in various medical applications.

### BACKGROUND

SpaceOAR®, a rapid crosslinking hydrogel that polymerizes in vivo within seconds, is based on a multi-arm polyethylene glycol (PEG) polymer functionalized with succinimidyl glutarate as activated end groups which further react with trilysine to form crosslinks. This product has become a very successful, clinically-used biomaterial in prostate cancer therapy. A further improvement based on this structure is that a portion the succinimidyl glutarate end groups have been functionalized with 2,3,5-triiiodobenzamide groups, providing radiopacity. This hydrogel, known by the trade name of SpaceOAR Vue®, is the radiopaque version of SpaceOAR® for prostate medical applications.

However, while the current synthetic route for forming the trilysine crosslinker is sufficient in that it produces the desired produce, the process, which is shown in FIG. 1, is very tedious as it involves five steps. Moreover, to the extent that additional lysine units may be desired (e.g., in the production of tetralysine, pentalysine, hexalysine, etc.), each added lysine requires an additional two steps.

For these and other reasons, alternative strategies are desired for forming trilysine and for forming iodine-labelled crosslinked hydrogels that provide enhanced radiopacity while maintaining crosslink density per polymer molecule.

### SUMMARY

In some aspects, the present disclosure pertains to methods that comprise (a) performing a ring-opening polymerization of one or more types of amino acid N-carboxyanhydride (NCA) monomers that comprise at least one type of protected amino acid NCA monomer having a protected pendant amine group in the presence of an initiator compound to produce intermediate peptide compounds that comprise an amino acid chain having protected pendant amine groups covalently attached to a residue of the initiator and (b) deprotecting the intermediate peptide compounds to form final peptide compounds that comprise an amino acid chain having pendant amine groups covalently attached to the residue of the initiator

In some embodiments, the method further comprises separating the final peptide compounds by molecular weight to provide final peptide compounds having amino acid chains of equal length.

In some embodiments, which can be used in conjunction with the above aspects and embodiments, a molar ratio of the amino acid N-carboxyanhydride (NCA) monomers to the initiator compound ranges from 2:1 to 100:1.

In some embodiments, which can be used in conjunction with the above aspects and embodiments, the amino acid chain ranges from 2 to 50 amino acid in length.

In some embodiments, which can be used in conjunction with the above aspects and embodiments, the protected pendant amine group is a protected primary amine group.

In some embodiments, which can be used in conjunction with the above aspects and embodiments, the protected pendant amine group is a protected alkylamine group.

In some embodiments, which can be used in conjunction with the above aspects and embodiments, the protected pendant amine group comprises a protective group selected from a tert-butoxycarbonyl group, a carboxybenzyl, a trifluoroacetyl group, a 6-nitroveratryloxycarbonyl group and a 9-fluorenylmethoxycarbonyl group.

In some embodiments, which can be used in conjunction with the above aspects and embodiments, the at least one type of protected amino acid NCA monomer comprises a protected lysine NCA monomer and/or a protected ornithine NCA monomer.

In some embodiments, which can be used in conjunction with the above aspects and embodiments, the initiator comprises a primary amine group or an aliphatic hydroxyl group.

In some embodiments, which can be used in conjunction with the above aspects and embodiments, the initiator comprises an amine protected by a trimethylsilyl group.

In some embodiments, which can be used in conjunction with the above aspects and embodiments, the initiator comprises (a) a primary amine group or an aliphatic group and (b) an iodinated aromatic group.

In some embodiments, which can be used in conjunction with the above aspects and embodiments, the initiator comprises (a) a primary amine group or an aliphatic group that is connected to (b) an iodinated aromatic group through a linear or multi-arm linker.

In some embodiments, which can be used in conjunction with the above aspects and embodiments, the initiator comprises an iodinated amino acid ester.

In some embodiments, which can be used in conjunction with the above aspects and embodiments, wherein the initiator comprises a metal catalyst.

In some embodiments, which can be used in conjunction with the above aspects and embodiments, the one or more types of amino acid N-carboxyanhydride (NCA) monomers comprise a single type of protected amino acid NCA monomer.

In some embodiments, which can be used in conjunction with the above aspects and embodiments, the one or more types of amino acid N-carboxyanhydride (NCA) monomers comprise at least one type of iodinated amino acid NCA monomer.

In some aspects, the present disclosure pertains to the final peptide compounds produced by a method in accordance with any of the above aspects and embodiments.

In some aspects, the present disclosure pertains to a system for forming a hydrogel that comprises (a) the final peptide compounds produced by a method in accordance with any of the above aspects and embodiments and (b) a reactive multi-arm polymer that comprises a plurality of hydrophilic polymer arms having reactive end groups that are reactive with amino groups of the final peptide compounds.

In some embodiments, the system further comprises a delivery device.

In some aspects, the present disclosure pertains to a reaction product of (a) the final peptide compounds produced by a method in accordance with any of the above aspects and embodiments and (b) a reactive multi-arm polymer that comprises a plurality of hydrophilic polymer arms having reactive end groups that are reactive with amino groups of the final peptide compounds.

In some aspects, the present disclosure pertains to methods of treatment comprising administering to a subject such a reaction product.

In some aspects, the present disclosure pertains to a method of treatment comprising administering to a subject a mixture that comprises the final peptide compounds produced by a method in accordance with any of the above aspects and embodiments and a reactive multi-arm polymer that comprises a plurality of hydrophilic polymer arms having reactive end groups that are reactive with amino groups of the final peptide compounds, under conditions such that the final peptide compounds and the reactive polymer crosslink after administration.

In some embodiments, the method of treatment comprises administering to the subject a first fluid composition that comprises the final peptide compounds and the reactive polymer and a second fluid composition that comprises an accelerant that accelerates formation of the covalent crosslinks. In certain embodiments, the first fluid composition and the second fluid composition are delivered using a double barrel syringe.

The above and other aspects, embodiments, features and benefits of the present disclosure will be readily apparent from the following detailed description.

### BRIEF DESCRIPTION OF THE DRAWINGS

FIG. 1 schematically illustrates a current scheme for forming trilysine.

FIG. 2 schematically illustrates a process for forming a peptide compound, in accordance with an embodiment of the present disclosure.

FIG. 3A schematically illustrates a process for forming a peptide compound, in accordance with an embodiment of the present disclosure.

FIG. 3B schematically illustrates a process for forming the initiator of FIG. 3A, in accordance with an embodiment of the present disclosure

FIG. 4A schematically illustrates a process for forming a peptide compound, in accordance with an embodiment of the present disclosure.

FIG. 4B schematically illustrates a process for forming the initiator of FIG. 4A, in accordance with an embodiment of the present disclosure.

FIG. 5A schematically illustrates a process for forming a peptide compound, in accordance with an embodiment of the present disclosure.

FIG. 5B schematically illustrates a process for forming the initiator of FIG. 5A, in accordance with an embodiment of the present disclosure

FIG. 6 schematically illustrates a process for forming a peptide compound, in accordance with an embodiment of the present disclosure.

FIG. 7 illustrates a delivery device, in accordance with an embodiment of the present disclosure.

FIG. 8 illustrates a delivery device, in accordance with another embodiment of the present disclosure.

### DETAILED DESCRIPTION

In some aspects of the present disclosure, peptide compounds are formed by ring-opening polymerization of amino acid N-carboxyanhydrides (NCAs) using a suitable initiator, which is incorporated into the peptide chain resulting from the polymerization. Peptide compounds in accordance with the present disclosure thus comprise an initiator residue and an amino acid chain that is covalently linked to the initiator residue. Initiator residues include amine-group-containing initiator residues and hydroxyl-group-containing residues. Amino acid chains may range from 2 to 50 amino acids in length, typically, from 3 to 10 amino acids in length. Amino acid chains in accordance with the present disclosure include amino acids having primary amine pendant groups. In particular examples, the primary amine pendant groups are aminoalkyl groups (e.g., C₁-C₆-aminoalkyl groups, including aminomethyl, 2-aminoethyl, 3-aminopropyl, 4-aminobutyl, 5-aminopentyl and 6-aminohexyl groups, as well as isomers of the same). The length of the amino acid chain is determined from the molar ratio of the amino acid NCA monomers to the initiator compound. In general, a molar ratio of the amino acid N-carboxyanhydride (NCA) monomers to the initiator compound ranges from 2:1 to 50:1, more typically from 3:1 to 10:1.

In some embodiments, amino acid NCA polymerization is conducted using an amine-group-containing initiator based on a nucleophilic ring opening chain growth process where the polymer grows linearly with monomer conversion. In this process, when the amine reacts with the NCA monomer, the NCA's ring opens, carbon dioxide is released, and a molecule with a new primary amine end group is formed, which is available for further reaction with another NCA monomer. The initiator that is used in this process is incorporated into the resulting peptide chain as previously noted. Additional information can be found, for example, in Carmen M. González-Henriquez, et al., “Strategies to Fabricate Polypeptide-Based Structures via Ring-Opening Polymerization of N-Carboxyanhydrides.” Polymers (Basel). 2017 November; 9(11): 551.

Initiators include those having an unmodified amine or a silyl protected amine, for example, a trimethylsilyl protected amine.

In the present disclosure, amino acid NCA monomers are employed that contain protected amine pendant groups. Examples of such amino acid NCA monomers include amine-protected lysine NCA monomers (where the protected group is a 4-aminobutyl group), and amine-protected ornithine NCA monomers (where the protected group is a 3-aminopropyl group), among others. Different protective chemistries have been reported for the case of lysine, with protective groups including tert-butoxycarbonyl (Boc) groups, carboxybenzyl (Cbz) or (Z) groups, trifluoroacetyl (TFA) groups, 6-nitroveratryloxycarbonyl (Nvoc) groups, and 9-fluorenylmethoxycarbonyl (Fmoc) groups. Id., citing Hernández, J. R.; Klok, H. A. “Synthesis and ring-opening (co) polymerization of L-lysine N-carboxyanhydrides containing labile side-chain protective groups.” J. Polym. Sci. Part A 2003, 41, 1167-1187.

Particular examples of amine-protected amino acid NCA monomers include

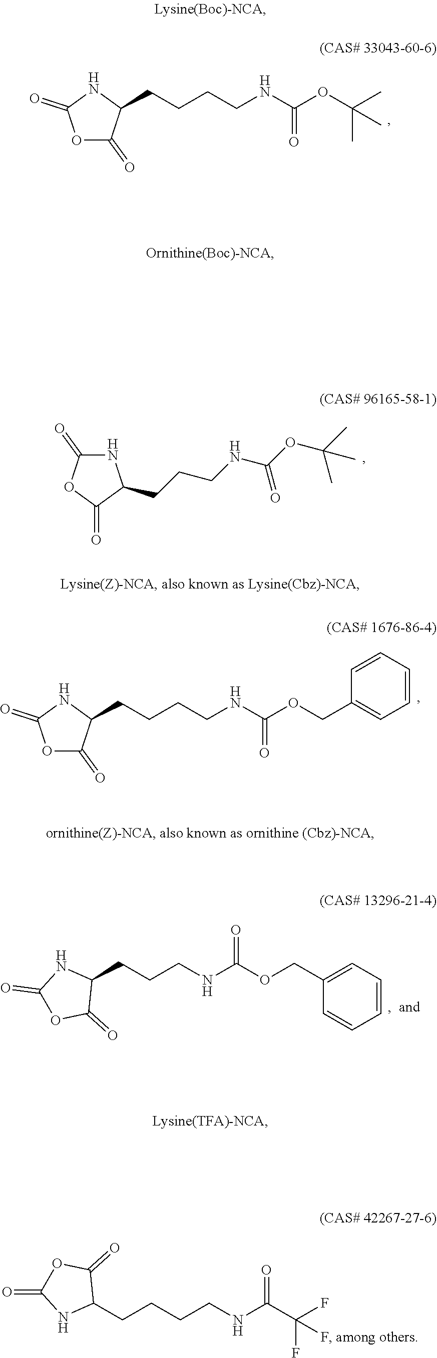

In the present disclosure, a wide range of primary and secondary amine initiators may be employed. In some embodiments, iodinated amine initiators may be employed to provide the resulting peptide with radiopacity. In some of these embodiments, the iodinated amine initiators are compounds that comprise a primary or secondary amine group, more typically a primary amine group, and one or more iodinated aromatic groups.

Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In some embodiments, the aromatic groups may be further substituted with one or more hydrophilic groups, for example, the aromatic groups may be further substituted with one, two, three, four, five, six or more hydrophilic groups. The one or more hydrophilic groups may comprise, for example, one or more of the following groups: hydroxyl groups, hydroxyalkyl groups (e.g., C₁-C₄-hydroxyalkyl groups containing one, two, three or four carbon atoms and containing one, two, three or four or more hydroxyl groups) and ester groups (e.g., C₂-C₆-ester groups containing two carbons, three carbons, four carbons, five carbons, six carbons, etc.) among others. The one or more hydrophilic groups may be linked to the aromatic group directly or through any suitable linking moiety, which may be selected, for example, from alkyl groups (e.g., alkyl groups containing one carbon, two carbons, three carbons, four carbons, etc.), amide groups, amine groups, ether groups, ester groups, or carbonate groups, among others.

Specific examples of iodinated amine initiators for use in the present disclosure include

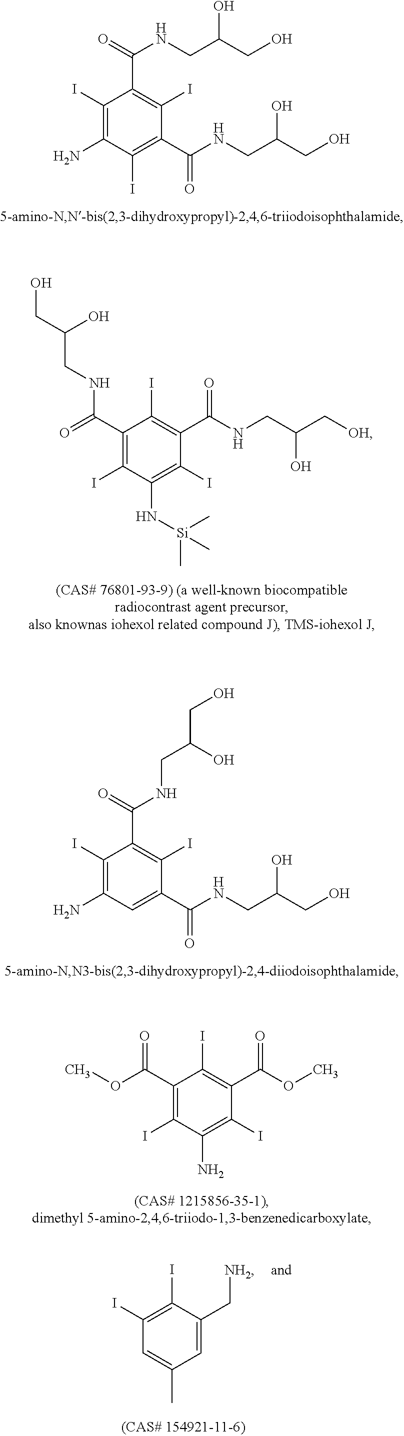

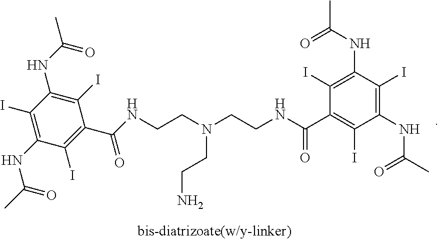 [SMILES: CC(=O)Nc1c(I)c(NC(C)=O)c(I)c(C(=O)NCCN(CCN)CCNC(=O)c2c(I)c(NC(C)=O)c(I)c(NC(C)=O)c2I)c1I]

Examples of iodinated amine initiators further include iodinated amino acid esters, for example, C₁-C₅-alkyl esters of iodinated amino acids, preferably methyl esters of iodinated amino acids. Particular examples include C₁-C₅-alkyl esters of any of the iodinated amino acids described below. After polymerization is complete, the C₁-C₅-alkyl ester may be converted into the corresponding carboxylic acid, if desired.

As used herein, an “amino acid” is an organic compound that contains an amino group (—NH₂), a carboxylic acid group (—COOH), and a side group that is specific to each amino acid. Depending on the surrounding pH, the amino group may be positively charged (—NH₃⁺) and/or the carboxylic acid group may be negatively charged (—COO⁻). An iodinated amino acid is an amino acid in which the side group contains one or more iodine atoms.

In various embodiments, the side group of the iodinated amino acid comprises one, two, three, four, five, six, seven, eight or more or more iodinated aromatic groups. The one or more iodinated aromatic groups may be directly linked to the remainder of the amino acid, linked to the remainder of the amino acid through a suitable linking moiety, which may be selected, for example, from alkyl groups (e.g., C₁-C₄-alkyl groups containing one carbon, two carbons, three carbons, four carbons, etc.), amide groups, amine groups, ether groups, ester groups, or carbonate groups, among others, or linked to another iodinated aromatic group through a suitable linking moiety, which may be selected, for example, from alkyl groups (e.g., C₁-C₄-alkyl groups containing one carbon, two carbons, three carbons, four carbons, etc.), amide groups, amine groups, ether groups, ester groups, or carbonate groups, among others. Examples of iodinated aromatic groups include iodine-substituted monocyclic aromatic groups and iodine-substituted multicyclic aromatic groups such as those set forth above.

Examples of iodinated amino acid esters include iodinated alpha-amino acid esters, iodinated beta-amino acid esters, iodinated gamma-amino acid esters, iodinated delta-amino acid esters, and iodinated epsilon-amino acid esters, among others.

Specific examples of iodinated amino acid ester initiators include the following:

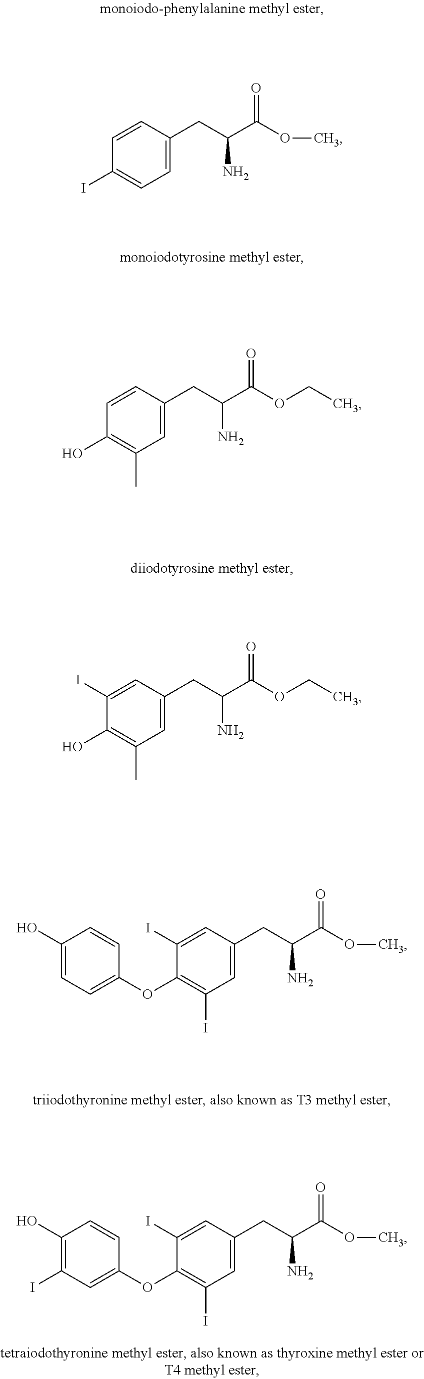 [SMILES: COC(=O)[C@@H](N)Cc1ccc(I)cc1 ; CCOC(=O)C(N)Cc1ccc(O)c(C)c1 ; CCOC(=O)C(N)Cc1cc(C)c(O)c(I)c1 ; COC(=O)[C@@H](N)Cc1cc(I)c(Oc2ccc(O)cc2)c(I)c1 ; COC(=O)[C@@H](N)Cc1cc(I)c(Oc2ccc(O)c(I)c2)c(I)c1]

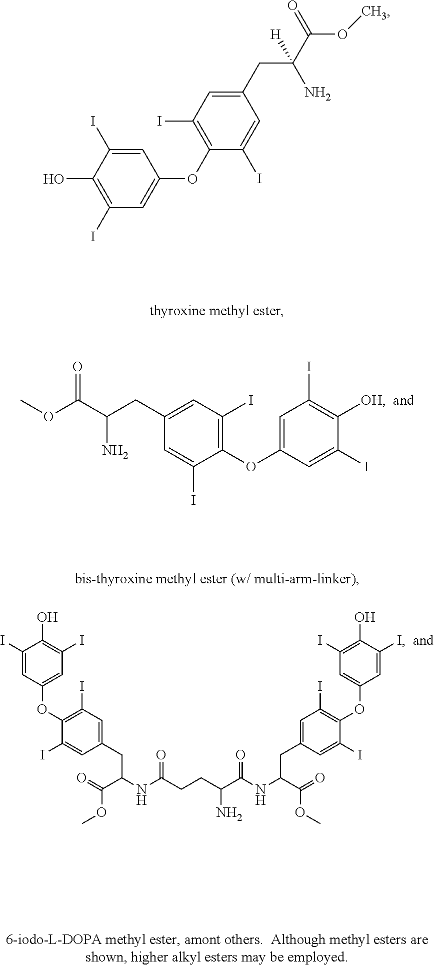

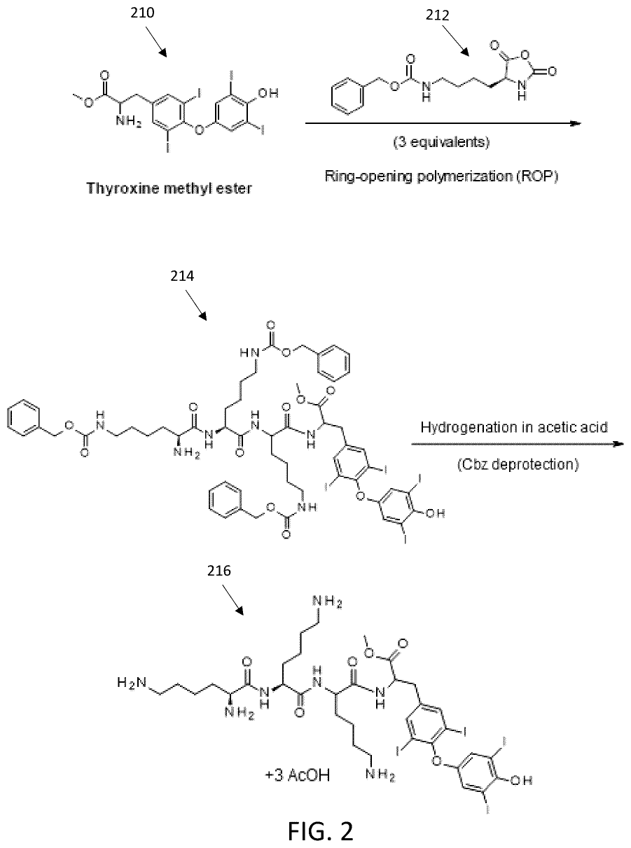

A particular example of a procedure in accordance with the present disclosure wherein a radiopaque peptide compound is formed by ring-opening polymerization of a protected amino acid NCA will now be described with reference to FIG. 2. In FIG. 2, a thyroxine methyl ester (210) is used as an iodinated amine initiator and Lysine(Cbz)-NCA (212) is used as an NCA monomer in a ring-opening polymerization step to form a protected radiopaque peptide compound (214) comprising a Cbz-protected trilysine amino acid chain covalently attached to a thyroxine methyl ester residue through an amide linkage. In a subsequent step, the protected radiopaque peptide compound (214) is deprotected by hydrogenation in acetic acid to form a final radiopaque peptide compound (216) comprising a trilysine amino acid chain covalently attached to a thyroxine methyl ester residue through an amide linkage.

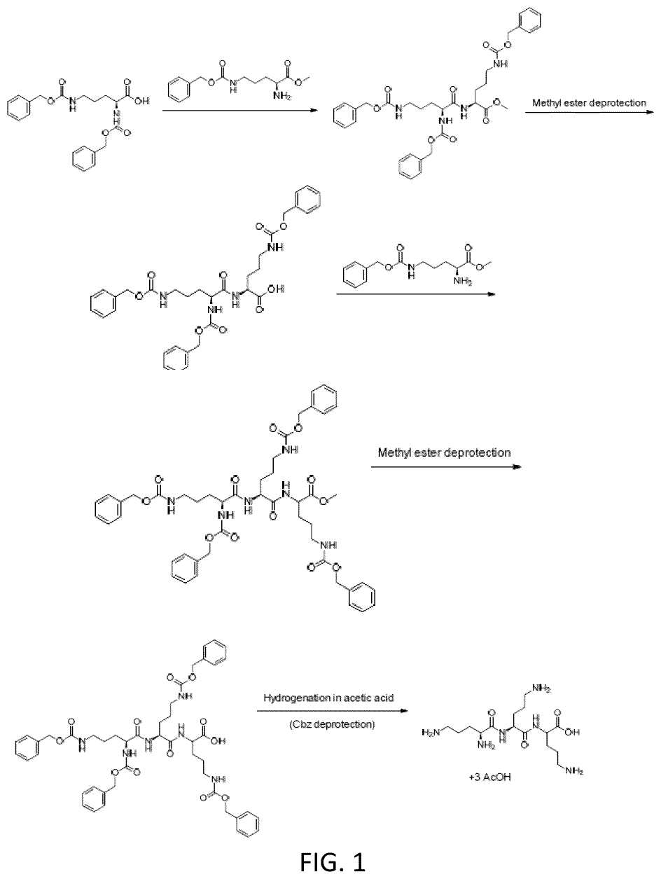

Because a tripeptide is being formed, a monomer-to-initiator ratio of about 3:1 is employed in the ring-opening polymerization of FIG. 2. For tetrapeptides, pentapeptides, hexapeptides, heptapeptides, octapeptides, nonapeptides, decapeptides, etc. respective monomer-to-initiator ratios will typically be about 4:1, about 5:1, about 6:1, about 7:1, about 8:1, about 9:1, about 10:1, etc. It will be appreciated that a major advantage of the peptide synthesis schemes of the present disclosure over that of FIG. 1 is that peptides of increasing length can be formed by increasing the monomer-to-initiator ratio without the need for any additional synthetic steps. As discussed further below, the peptide products of the present disclosure have utility as crosslinking agents due to the presence of primary amine groups. The presently described synthesis procedure allows the number of primary amine crosslinking groups to be tuned without the need for additional synthetic steps, which would otherwise increase the cost of the crosslinking agents.

It is noted that the products of the ring-opening polymerization processes described herein will be statistical products meaning that peptides of varying length will be produced. For example, in the case of the above lysine-thyroxine conjugates, a majority of the product with be the trilysine-thyroxine conjugate, however, other conjugates including dilysine-thyroxine conjugate and tetralysine-thyroxine conjugate, are expected to be present. Therefore, in various embodiments, the products of the ring-opening polymerization processes described herein may be subjected to a further purification step to separate the desired peptide product (in this case the trilysine product) from products of differing length (which will have a lower or higher molecular weight). Techniques for separating compounds based on their molecular weights include high-performance liquid chromatography (HPLC), simulated moving bed chromatography, ion exchange separations, and membrane filtration, among others.

It is also noted that the alkyl ester products formed herein can be converted to carboxylic acid products by hydrolysis.

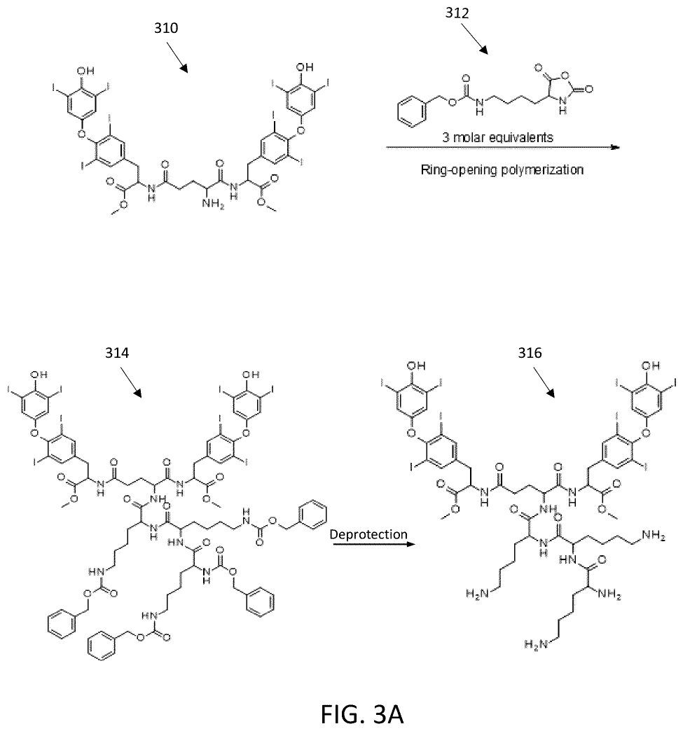

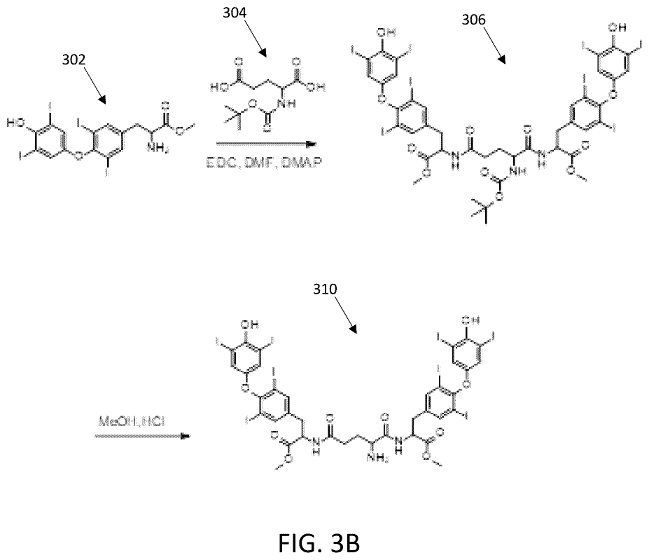

FIG. 3A illustrates another example of a procedure in accordance with the present disclosure wherein bis-thyroxine methyl ester (w/multi-arm linker) (310) is used as the iodinated amine initiator and Lysine(Cbz)-NCA (312) is used as the NCA monomer in a ring-opening polymerization step to form a protected radiopaque peptide compound (314) comprising a Cbz-protected trilysine amino acid chain covalently attached to a bis-thyroxine residue through an amide linkage. A glutamic acid multi-arm linker is shown here as an example, but any linear or multi-arm linker may be employed. In a subsequent step, the protected radiopaque peptide compound (314) is deprotected by hydrogenation in acetic acid to form a final radiopaque peptide compound (316) comprising a trilysine amino acid chain covalently attached to a bis-thyroxine methyl ester residue through an amide linkage.

FIG. 3B illustrates a procedure in accordance with the present disclosure for the formation of the bis-thyroxine methyl ester compound (310) of FIG. 3A. As shown in FIG. 3B, thyroxine methyl ester (302), 1-ethyl-3-(3-dimethyl'propyl)carbodiimide (EDC) coupling agent, t-boc protected alpha glutamic acid (304) and a catalytic amount of 4-dimethylaminopyridine (DMAP) are dissolved in dimethylformamide (DMF). The reaction mixture is stirred at 50° C. for 24 hours. After the coupling reaction, the solvent is removed in vacuo, and the t-boc protected product (306) dissolved in ethyl acetate and washed with brine. The ethyl acetate is then removed in vacuo. The t-boc protected product (306) is dissolved in methanol and deprotected by acidification with HCl. The solution is stirred at room temperature overnight. After the reaction, the solvent is removed in vacuo to yield the bis-thyroxine methyl ester (w/multi-arm linker) (310).

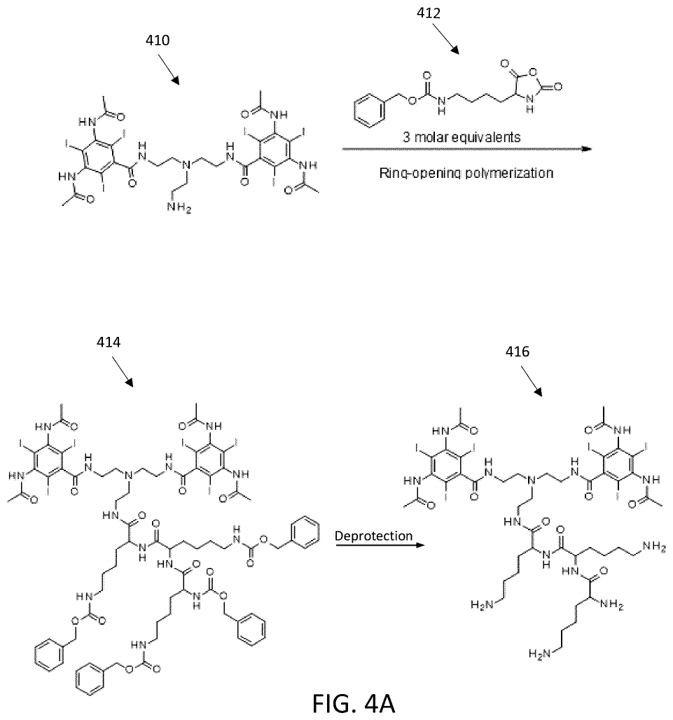

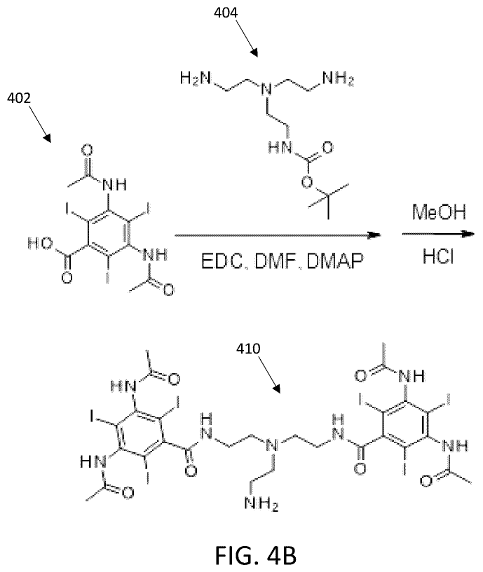

FIG. 4A illustrates a further example of a procedure in accordance with the present disclosure wherein bis-diatrizoate (w/multi-arm-linker) (410) is used as the iodinated amine initiator and Lysine(Cbz)-NCA (412) is used as the NCA monomer in a ring-opening polymerization step to form a protected radiopaque peptide compound (414) comprising a Cbz-protected trilysine amino acid chain covalently attached to a bis-diatrizoate residue through an amide linkage. In a subsequent step, the protected radiopaque peptide compound (414) is deprotected by hydrogenation in acetic acid to form a final radiopaque peptide compound (416) comprising a trilysine amino acid chain covalently attached to a bis-methyl ester residue through an amide linkage.

FIG. 4B illustrates a procedure in accordance with the present disclosure for the formation of the bis-diatrizoate compound (410) of FIG. 4A. As shown in FIG. 4B, diatrizoate (402), t-boc protected tris-2-aminoethylamine (404), EDC coupling agent, and a catalytic amount of DMAP are dissolved in DMF. The reaction mixture is stirred at 50° C. for 24 hours. After the coupling reaction, the solvent is removed in vacuo, and the t-boc protected product (not shown) is dissolved in ethyl acetate and washed with brine. The ethyl acetate is then removed in vacuo. The t-boc protected product is dissolved in methanol and acidified with HCl. The solution is stirred at room temperature overnight. After the reaction, the solvent is removed in vacuo to yield the bis-diatrizoate (w/multi-arm-linker) (410).

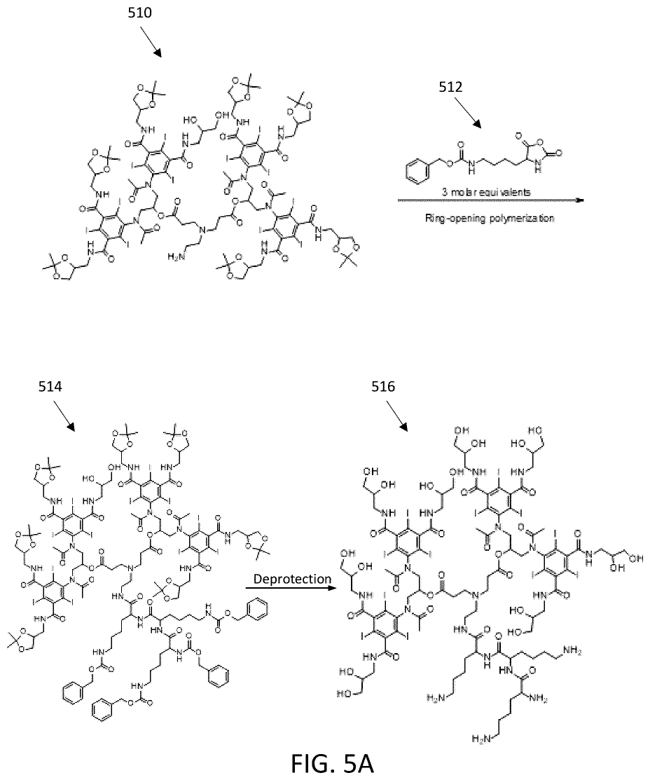

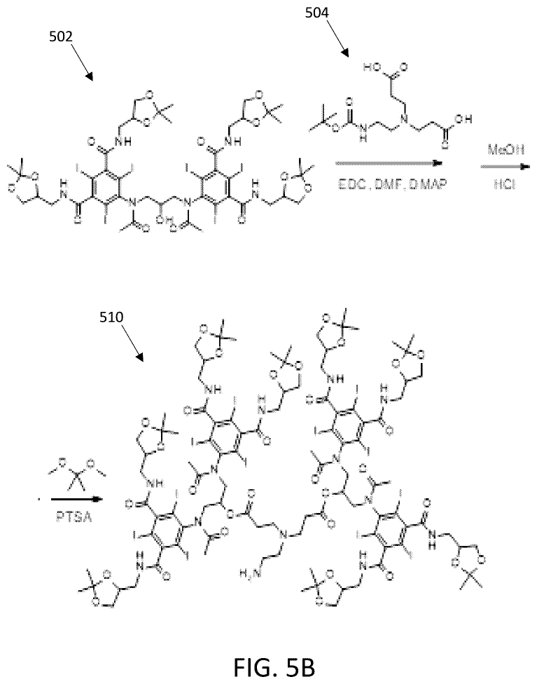

FIG. 5A illustrates a further example of a procedure in accordance with the present disclosure wherein acetal-protected bis-iodixanol (w/multi-arm-linker) (510) is used as the iodinated amine initiator and Lysine(Cbz)-NCA (512) is used as an NCA monomer in a ring-opening polymerization step to form a radiopaque acetal- and Cbz-protected radiopaque peptide compound (514). In a subsequent step, the radiopaque acetal- and Cbz-protected radiopaque peptide compound (514) is deprotected to form a final radiopaque trilysine product (516) in which a trilysine oligomer is linked to a bis-iodixanol residue through an amide linkage.

FIG. 5B illustrates a procedure in accordance with the present disclosure that can be used for the formation of the bis-iodixanol compound of FIG. 4A. As shown in FIG. 4B, acetal protected iodixanol (502), t-boc protected 3-[(2-aminoethyl)(2-carboxyethyl)amino]propanoic acid dihydrochloride (504) (CAS of parent compound: 141702-92-3), EDC coupling agent and a catalytic amount of DMAP are dissolved in DMF. The reaction mixture is stirred at 50° C. for 24 hours. After the coupling reaction, the solvent is removed in vacuo, and the product (not shown) is dissolved in ethyl acetate and washed with brine. The ethyl acetate is then removed in vacuo. The product is dissolved in methanol and acidified with HCl. The solution is stirred at room temperature overnight. After the reaction, the solvent is removed in vacuo to yield a bis-iodixanol (w/multi-arm-linker) compound (516), which is the deprotected version of the bis-iodixanol (w/multi-arm-linker) compound (510).

In other embodiments of the present disclosure, amino acid NCA polymerization is initiated by a hydroxyl-group-containing initiator. This polymerization is performed by acid catalyzed initiation, followed by base quenching and polymerization, with the acid catalyst being methane sulfonic acid. Base quenching can be accomplished with N-ethyldiisopropylamine, or triethylamine.

In the present disclosure, a wide range of hydroxyl-group-containing initiators may be employed. In some embodiments, iodinated hydroxyl-group-containing initiators may be employed to provide the resulting peptide with radiopacity. In some of these embodiments, the hydroxyl-group-containing initiators are compounds that comprise a hydroxyl group and one or more iodinated aromatic groups. Examples of iodinated aromatic groups include iodine-substituted monocyclic aromatic groups and iodine-substituted multicyclic aromatic groups such as those set forth above. In some embodiments, the one or more hydroxyl groups are found in hydroxyl-group-containing ring substituents of the iodine-substituted monocyclic aromatic groups or the iodine-substituted multicyclic aromatic groups. For example, the iodine-substituted monocyclic aromatic groups and/or the iodine-substituted multicyclic aromatic groups may be substituted with one or more C₁-C₆-hydroxyalkyl groups. Where two or more hydroxyl groups are present on the initiator, a branched peptide may be formed.

A particular example of a hydroxyl-group-containing initiator is iodixanol (which is a well-known biocompatible radiocontrast agent precursor),

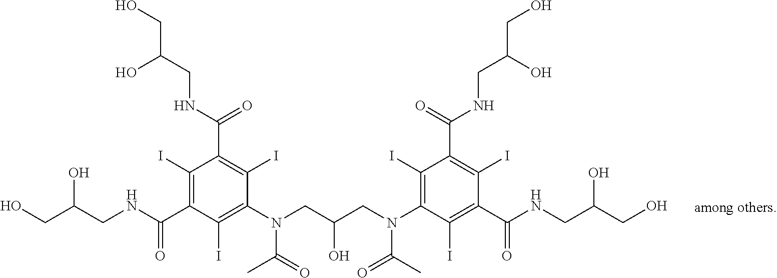 [SMILES: CC(=O)N(CC(O)CN(C(C)=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I]

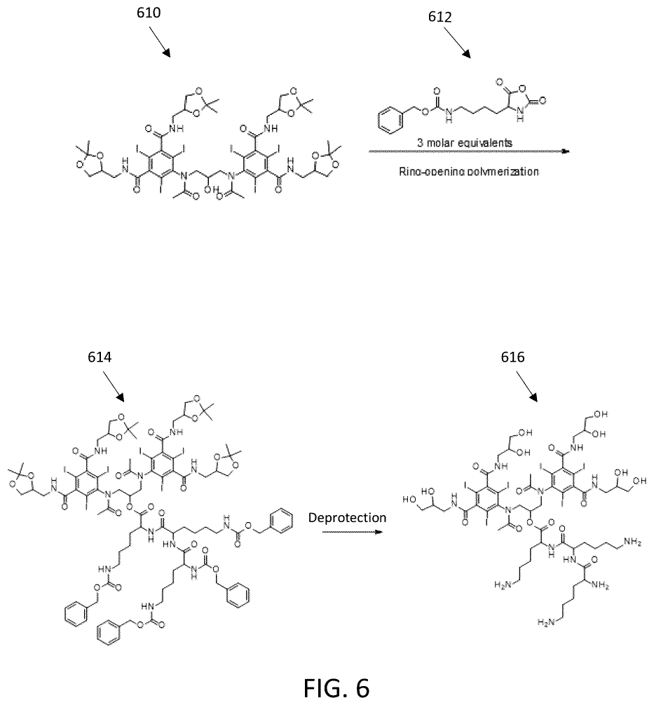

FIG. 6 illustrates a further example of a procedure in accordance with the present disclosure wherein acetal-protected iodixanol (610; CAS: 192449-65-3, synthesized from parent iodixanol) is used as an iodinated hydroxyl-group-containing initiator and Lysine(Cbz)-NCA (612) is used as an NCA monomer in a ring-opening polymerization step to form a radiopaque acetal- and Cbz-protected radiopaque peptide compound (614). In a subsequent step, the radiopaque acetal- and Cbz-protected radiopaque peptide compound (614) is deprotected via acetal deprotection (iodine chloride followed by sodium hydroxide) and Cbz deprotection with Pd(OAc)₂ with a hydrogen source, or mercaptoethanol, to form a final radiopaque trilysine product (616) in which a trilysine oligomer is linked to an iodixanol residue through an ester linkage.

In the case iodixanol, it noted that an alternative scheme is possible in which the central hydroxyl group of the iodixanol is reacted, for example, with a diamine compound to provide a primary amine group that is linked to an iodixanol by an amide-based linker.

It is noted that, although Cbz protection of the lysine NCA is shown in FIGS. 2-6, other types of protection, including the protective groups described above, may be employed. See, e.g., Brian V. Falcone, et al., “Synthesis of bis-Phenylalanine, A Novel Eight-Membered Cyclic Dipeptide” Synthetic Communications, 38: 411-418, 2008, which describes Boc deprotection in the presence of aryl iodides, specifically, Scheme 1, wherein condition a accomplishes Boc deprotection, condition b leads to the FMOC protection, with 4N HCl in dioxane unveiling the deprotected amine.

It is also noted that although iodine groups are specifically described herein, other radiopaque halogen groups including bromine may be employed.

It is further noted that, although the above-described initiators are iodinated initiators, non-iodinated initiators are useful in the present disclosure as well. Particular examples of non-iodinated initiators include ammonium chloride, hexamethyldisilazane (CAS 999-97-3), which, upon deprotection by a fluoride source, would yield trilysine, and other aliphatic amines, or trimethylsilyl protected aliphatic amines. Additionally, transition metal initiators can be used to polymerize the NCA monomers. These transition metal complexes can include Cobalt, Nickel, etc., and can be removed via precipitation or dialysis after polymerization

In other embodiments, radiopacity can be introduced into the final product by using iodinated amino acid NCA derivatives in the ring-opening synthesis. For example, protected iodinated phenylalanine NCA or protected iodinated tyrosine NCA may be used in the ring-opening synthesis in some embodiments. These could be used to form statistical copolymers, gradient copolymers, or block copolymers with the protected amine containing NCA derivatives.

In some aspects of the present disclosure, crosslinked hydrogels are provided that comprises a crosslinked reaction product of (a) a peptide compound in accordance with the present disclosure that comprises an initiator residue and an amino acid chain comprising a plurality of amino acids having primary amine pendant groups and (b) a reactive polymer comprising reactive moieties.

Such crosslinked hydrogels may be formed in vivo (e.g., using a delivery device like that described below), or such crosslinked hydrogels may be formed ex vivo and subsequently administered to a subject. Such crosslinked hydrogels can be used in a variety of biomedical applications, including implants, medical devices, and pharmaceutical compositions.

In some embodiments, the crosslinked hydrogel is visible under fluoroscopy. The crosslinked hydrogel may have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values).

Reactive polymers for use in the present disclosure include reactive multi-arm polymers that comprise a plurality of polymer arms linked to a core region, at least a portion of the arms comprising a hydrophilic polymer segment. One end of the hydrophilic polymer segment is covalently linked to the core region and an opposite end of the hydrophilic polymer segment is covalently linked to a reactive moiety.

In certain embodiments, at least a portion of the polymer arms comprise a hydrophilic polymer segment that has first and second ends, the first end of the hydrophilic polymer segment covalently linked to the core region, a cyclic anhydride residue having first and second ends, the first end of the cyclic anhydride residue covalently linked to the second end of the hydrophilic polymer segment, and a reactive moiety that is covalently linked to the second end of the cyclic anhydride residue.

Reactive polymers in accordance with the present disclosure include polymers having from 3 to 100 arms, for example ranging anywhere from 3 to 4 to 5 to 6 to 7 to 8 to 10 to 12 to 15 to 20 to 25 to 50 to 75 to 100 arms (in other words, having a number of arms ranging between any two of the preceding values).

Reactive moieties include moieties that comprise electrophilic groups.

Electrophilic groups may be selected, for example, from cyclic imide ester groups, such as succinimide ester groups,

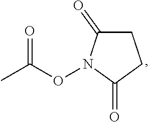

maleimide ester groups, glutarimide ester groups, diglycolimide ester groups, phthalimide ester groups, and bicyclo[2.2.1]hept-5-ene-2,3-dicarboxylic acid imide ester groups,

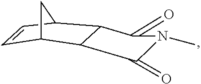

imidazole ester groups, imidazole carboxylate groups and benzotriazole ester groups, among other possibilities.

The electrophilic groups may be linked to the hydrophilic polymer segment through any suitable linking moiety, which may be selected, for example, from a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, or a linking moiety that comprises a combination of two or more of the foregoing groups, among others. In certain embodiments, the linking moiety comprises a hydrolysable ester group.

Hydrophilic polymer segments for the polymer arms can be selected from a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain between 10 and 1000 monomer units or more.

In certain embodiments, the core region comprises a residue of a polyol comprising three or more hydroxyl groups, which is used to form the polymer arms. In certain beneficial embodiments, the core region comprises a residue of a polyol that contains from 3 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

In other embodiments, the core region comprises a silsesquioxane, which is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three 0 atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

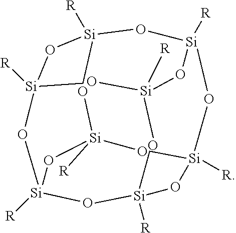

In the present disclosure, the R groups comprise the polymer arms described herein.

Reactive multi-arm polymers in accordance with the present disclosure can be formed from hydroxy-terminated precursor multi-arm polymers having arms that comprise one or more hydroxyl end groups. In some of these embodiments, the hydroxy-terminated precursor multi-arm hydrophilic polymer may be reacted with a cyclic anhydride to form an acid-end-capped precursor polymer. For example, terminal hydroxyl groups of the hydrophilic segments may be reacted with a cyclic anhydride (e.g., a glutaric anhydride compound, a succinic anhydride compound, a malonic anhydride compound, an adipic anhydride compound, a diglycolic anhydride compound, etc.) to form an acid-end-capped segment such as a glutaric-acid-end-capped segment, a succinic-acid-end-capped segment, a malonic-acid-end-capped segment, an adipic-acid-end-capped segment, a diglycolic-acid-end-capped segment, and so forth.

The preceding cyclic anhydrides, among others, may be reacted with a hydroxy-terminated precursor multi-arm hydrophilic polymer under basic conditions to form a carboxylic-acid-terminated precursor polymer comprising a carboxylic acid end group that is linked to a hydrophilic polymer segment through a hydrolysable ester group.

A reactive moiety may then be linked to the carboxylic-acid-terminated precursor polymer.

In some embodiments, an electrophilic moiety may be linked to the carboxylic-acid-terminated precursor polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl'propyl)carbodiimide (EDC), N-hydroxybenzotriazole (HOBt), BOP reagent, and/or another coupling agent) to form a reactive cyclic imide ester (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to a hydrophilic polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

In some aspects of the present disclosure, a system is provided that comprises (a) a first composition that comprises a peptide compound as described herein and (b) a second composition that comprises a reactive polymer comprising reactive moieties as described herein, wherein the system is configured to deliver the reactive polymer and the peptide compound under conditions such that covalent crosslinks are formed between the reactive polymer and the peptide compound.

The first composition may be a first fluid composition comprising the peptide compound or a first dry composition that comprises the peptide compound, to which a suitable fluid such as water for injection, saline, etc. can be added to form a first fluid composition. In addition to the peptide compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer, to which a suitable fluid such as water for injection, saline, etc. can be added to form a second fluid composition. In addition to the reactive polymer, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the peptide compound with a second fluid comprising the reactive polymer. Upon mixing the first and second fluid compositions, the peptide compound crosslink with the reactive polymer, forming a crosslinked product. The first and second fluid compositions may be combined form crosslinked hydrogels, either in vivo or ex vivo.

In some embodiments, the peptide compound is initially combined with the reactive polymer under conditions where crosslinking between the reactive polymer and the peptide compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between the peptide compound and the reactive polymer, thereby forming a crosslinked product.

In some embodiments, the system comprises (a) a first composition that comprises peptide compound as described hereinabove, (b) a second composition that comprises a reactive polymer as described hereinabove, and (c) a third composition, specifically, an accelerant composition, that contains an accelerant that is configured to accelerate a crosslinking reaction between the peptide compound and the reactive polymer.

The first composition may be a first fluid composition comprising the peptide compound that is buffered to an acidic pH or a first dry composition that comprises the peptide compound, to which a suitable fluid such as water for injection, saline, an acidic buffer solution, etc. can be added to form a first fluid composition comprising the peptide compound that is buffered to an acidic pH. In some embodiments, for example, the acidic buffering composition may comprise monobasic sodium phosphate, among other possibilities. The first fluid composition comprising the peptide compound may have a pH ranging, for example, from about 3 to about 5. In addition to the peptide compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer from which a fluid composition is formed, for example, by the addition of a suitable fluid such as water for injection, saline, or the first fluid composition comprising the peptide compound that is buffered to an acidic pH. In addition to the reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the peptide compound that is buffered to an acidic pH and the second composition comprises a dry composition that comprises the reactive polymer. The first composition may then be mixed with the second composition to provide a prepared fluid composition that is buffered to an acidic pH and comprises the peptide compound and the reactive polymer. In a particular example, a syringe may be provided that contains the first fluid composition comprising the peptide compound that is buffered to an acidic pH, and a vial may be provided that comprises the dry composition (e.g., a powder) that comprises the reactive polymer. The syringe may then be used to inject the first fluid composition into the vial containing the reactive polymer to form a prepared fluid composition that is buffered to an acidic pH and contains the peptide compound and the reactive polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

A prepared fluid composition that is buffered to an acidic pH and comprises the peptide compound and the reactive polymer as described above, and a fluid accelerant composition that is buffered to basic pH as described above, may be combined form crosslinked hydrogels, either in vivo or ex vivo.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethene (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises a peptide compound as described herein and a second reservoir that contains a second fluid composition that comprises a reactive polymer as described herein, wherein the first and second fluid compositions form a crosslinked product upon mixing. In some embodiments, the system may include a delivery device that comprises a first reservoir that contains a first fluid composition that comprises the peptide compound and the reactive polymer and is buffered to an acidic pH, such as the prepared fluid composition previously described, and a second reservoir that contains second fluid composition, such as the fluid accelerant composition previously described.

In either case, during operation, the first fluid composition and second fluid composition are dispensed from the first and second reservoirs and combined, whereupon the peptide compound and the reactive polymer and crosslink with one another to form a crosslinked hydrogel.

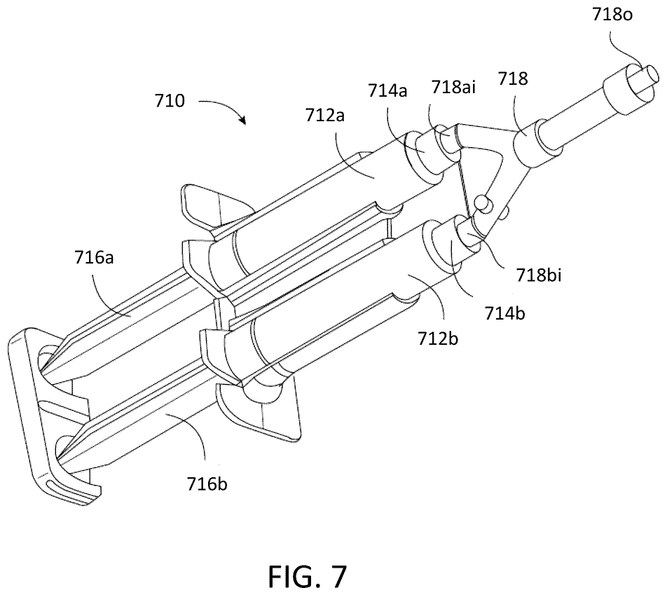

In particular embodiments, and with reference to FIG. 7, the system may include a delivery device 710 that comprises a double-barrel syringe, which includes a first barrel 712a having a first barrel outlet 714a, which first barrel contains the first composition, a first plunger 716a that is movable in the first barrel 712a, a second barrel 712b having a second barrel outlet 714b, which second barrel 712b contains the second composition, and a second plunger 716b that is movable in the second barrel 712b. In some embodiments, the device 710 may further comprise a mixing section 718 having a first mixing section inlet 718ai in fluid communication with the first barrel outlet 714a, a second mixing section inlet 718bi in fluid communication with the second barrel outlet, and a mixing section outlet 718o.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique. Typically, the imaging techniques is an x-ray-based imaging technique, such as computerized tomography or x-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-releasing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may varying widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the crosslinked hydrogel composition to a subject. Such systems may include one or more of the following: a syringe barrel, which may or may not contain a crosslinked hydrogel composition as described herein; a vial, which may or may not contain a crosslinked hydrogel composition as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel composition may be provided in dry form (e.g., powder form) or in a form that is ready for injection, such as an injectable hydrogel form (e.g., a suspension of crosslinked hydrogel particles).

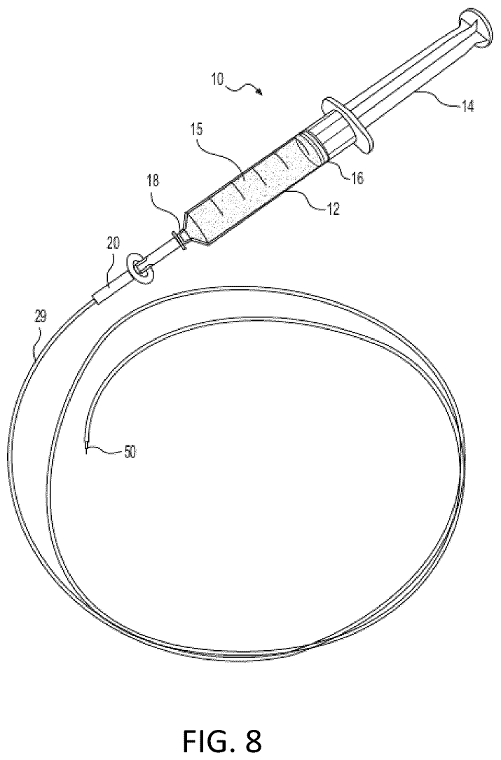

FIG. 8 illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

After administration, the crosslinked hydrogel compositions of the present disclosure can be imaged using a suitable imaging technique.

As seen from the above, the crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A method comprising (a) performing a ring-opening polymerization of one or more types of amino acid N-carboxyanhydride (NCA) monomers that comprise at least one type of protected amino acid NCA monomer having a protected pendant amine group in the presence of an initiator compound to produce intermediate peptide compounds that comprise an amino acid chain having protected pendant amine groups covalently attached to a residue of the initiator and (b) deprotecting the intermediate peptide compounds to form final peptide compounds that comprise an amino acid chain having pendant amine groups covalently attached to the residue of the initiator.

2. The method of claim 1, further comprising separating the final peptide compounds by molecular weight to provide final peptide compounds having amino acid chains of equal length.

3. The method of claim 1, wherein a molar ratio of the amino acid N-carboxyanhydride (NCA) monomers to the initiator compound ranges from 2:1 to 100:1.

4. The method of claim 1, wherein the amino acid chain ranges from 2 to 50 amino acid in length.

5. The method of claim 1, wherein the protected pendant amine group is a protected primary amine group.

6. The method of claim 1, wherein the protected pendant amine group is a protected alkylamine group.

7. The method of claim 1, wherein the protected pendant amine group comprises a protective group selected from a tert-butoxycarbonyl group, a carboxybenzyl, a trifluoroacetyl group, a 6-nitroveratryloxycarbonyl group and a 9-fluorenylmethoxycarbonyl group.

8. The method of claim 1, wherein the at least one type of protected amino acid NCA monomer comprises a protected lysine NCA monomer and/or a protected ornithine NCA monomer.

9. The method of claim 1, wherein the initiator comprises a primary amine group or an aliphatic hydroxyl group.

10. The method of claim 1, wherein the initiator comprises an amine protected by a trimethylsilyl group.

11. The method of claim 1, wherein the initiator comprises (a) a primary amine group or an aliphatic group and (b) an iodinated aromatic group.

12. The method of claim 1, wherein the initiator comprises (a) a primary amine group or an aliphatic group that is connected to (b) an iodinated aromatic group through a linear or multi-arm linker.

13. The method of claim 1, wherein the initiator comprises an iodinated amino acid ester.

14. The method of claim 1, wherein the one or more types of amino acid N-carboxyanhydride (NCA) monomers comprise a single type of protected amino acid NCA monomer.

15. The method of claim 1, wherein the one or more types of amino acid N-carboxyanhydride (NCA) monomers comprise at least one type of iodinated amino acid NCA monomer.

16. A system for forming a hydrogel that comprises (i) a reactive multi-arm polymer that comprises a plurality of hydrophilic polymer arms having reactive end groups and (ii) final peptide compounds produced by a method comprising (a) performing a ring-opening polymerization of one or more types of amino acid N-carboxyanhydride (NCA) monomers that comprise at least one type of protected amino acid NCA monomer having a protected pendant amine group in the presence of an initiator compound to produce intermediate peptide compounds that comprise an amino acid chain having protected pendant amine groups covalently attached to a residue of the initiator and (b) deprotecting the intermediate peptide compounds to form the final peptide compounds, which comprise an amino acid chain having pendant amine groups covalently attached to the residue of the initiator, wherein the reactive end groups are reactive with amino groups of the final peptide compounds.

17. The system of claim 16, further comprising a delivery device.

18. A method of treatment comprising administering to a subject a mixture that comprises (i) a reactive multi-arm polymer that comprises a plurality of hydrophilic polymer arms having reactive end groups and (ii) final peptide compounds produced by a method comprising (a) performing a ring-opening polymerization of one or more types of amino acid N-carboxyanhydride (NCA) monomers that comprise at least one type of protected amino acid NCA monomer having a protected pendant amine group in the presence of an initiator compound to produce intermediate peptide compounds that comprise an amino acid chain having protected pendant amine groups covalently attached to a residue of the initiator and (b) deprotecting the intermediate peptide compounds to form the final peptide compounds, which comprise an amino acid chain having pendant amine groups covalently attached to the residue of the initiator, wherein the reactive end groups are reactive with amino groups of the final peptide compounds, and wherein the mixture is administered under conditions such that the final peptide compounds and the reactive polymer crosslink after administration.

19. The method of claim 18, wherein the method comprises administering to the subject a first fluid composition that comprises the final peptide compounds and the reactive polymer and a second fluid composition that comprises an accelerant that accelerates formation of the covalent crosslinks.

20. The method of claim 19, wherein the first fluid composition and the second fluid composition are delivered using a double barrel syringe.
